(19) United States
(12) Patent Application Publication
(10) Pub. No.: US 2020/0215217 A1
(43) Pub. Date: Jul. 9, 2020

(54) Automatic Timer for Dispenser

(71) Applicant: Teppo Kullervo Jokinen, Mukilteo, WA (US)

(72) Inventor: Teppo Kullervo Jokinen, Mukilteo, WA (US)

(21) Appl. No.: 16/241,976

(22) Filed: Jan. 7, 2019

(51) Int. Cl. A61L 2/18; A61L 2/24

(52) U.S. Cl. CPC A61L 2/18; F24F 2003/1664; A61L 2/24

(57) ABSTRACT

Automatic timer for air conditioning condensate water chemical dispenser. This timer regulate automatically the time when chemicals are needed.

### BACKGROUND OF THE INVENTION

Air conditioning condensate water lines has tendency grow bacteria. For a short period of time, condensate water line is clogged by the bacteria. Several attempt has been made to stop the growth of bacteria, manually and by devises. The devices are controlled by a timers. The timers work year around regardless if treatment is needed or not.

### SUMMARY OF THE INVENTION

This timer for dispenser is automatic. The dispenser is activated only when it is needed to deliver decided chemical.

### BRIEF DESCRIPTION OF THE DRAWINGS

The dispenser have existing power source (20), thermostat (21) and dispenser mechanism. Power from power source (20) flows to delay on make timer (22), malty functional relay (23) and solenoid (24). Solenoid (24) is activated according to pre-set settings. When delay on make timer is energized, and terminals (5) and (6) are normally closed, solenoid (24) is energized and rod (42) will arise up position. Terminals (5) and (6) opens for pre-set time limit. The solenoid (24) is de-energized, rod (42) drops down to rest position

### DETAILED DESCRIPTION OF THE DRAWINGS

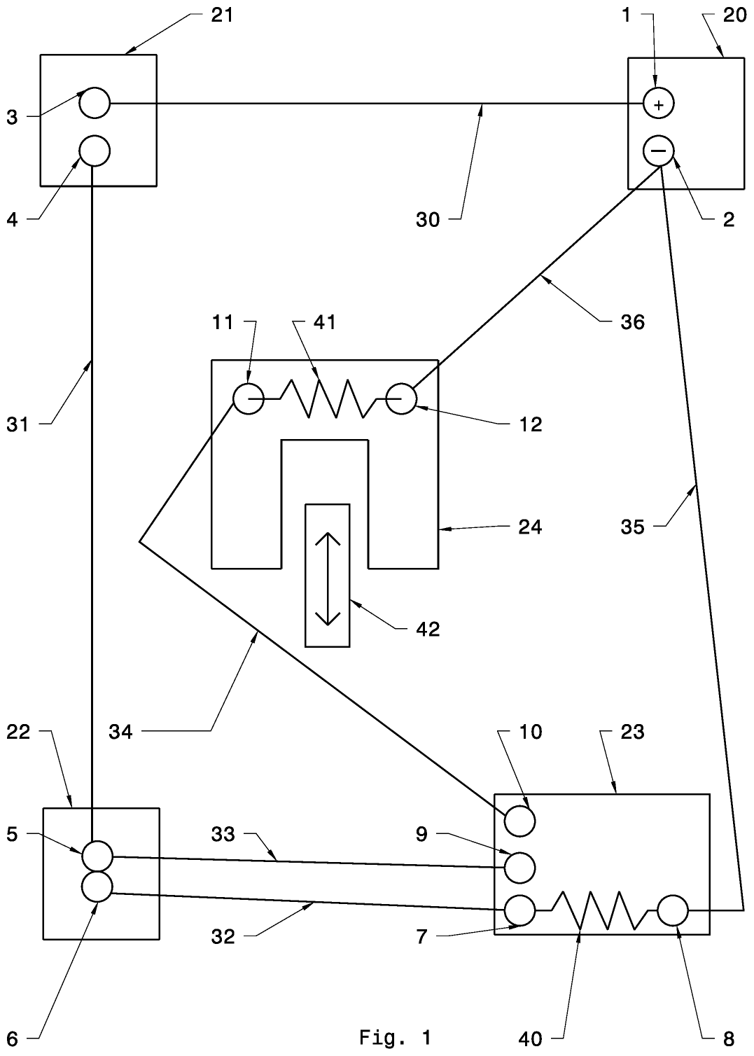

FIG. 1/3

Thermostat (21) not calling cooling. Current Cows from power source (20) to Terminal (1) to thermostat (21) terminal (3) thermostat (21) terminals (3) and (4) are open. Rest of the device is de-energized. There is no current Cow between terminals (5) and (6), (7) and (8), (10) and (9), (11) and (12), (12) and (2). The solenoid (24) rod (42) is down on a rest position.

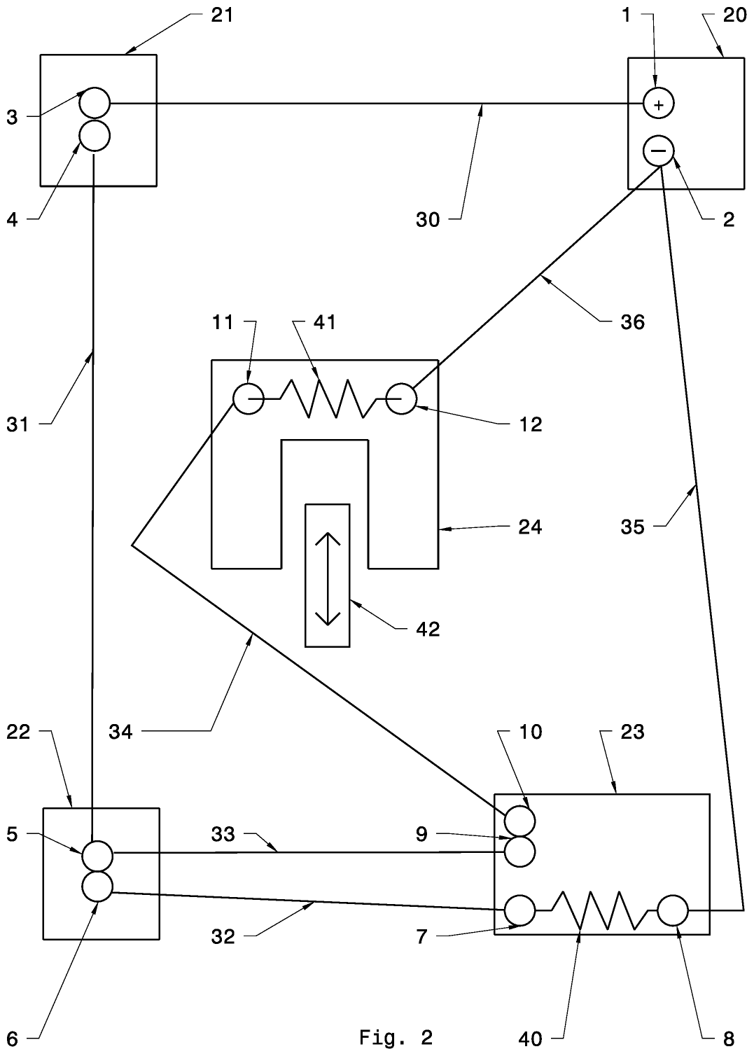

FIG. 2/3

Thermostat calling cooling. Current Cows power source (20) terminal (1) to thermostat (21) terminal (3). Thermostat (21) terminals (3) and (4) make a contact. Current flows from terminal (4) through wire (31) to delay on make timer (22), Terminal (5). Current flows from timer (22) terminal (5) to terminal (6). Contact is normally closed. Current flows from terminal (6) to terminal (7) on malty functional relay (23) through wire (32). Relay (23), current flows from terminal (7) through coil (40) to terminal (8). Current flows from terminal (8) to terminal (2) on power source(20), Current flows from timer (22) terminal (9) on relay (23). Terminals (9) and (10) are now closed. Current from relay (23) terminal (10) flows through wire (34) to terminal (11) on solenoid (24). Current, solenoid (24) flows from terminal (11) through coil (41) to terminal (12). Current from terminal (12) on solenoid (24) flows through wire (36) to power source (20) to terminal (2). On the solenoid (24) the rod (42) is now in up position

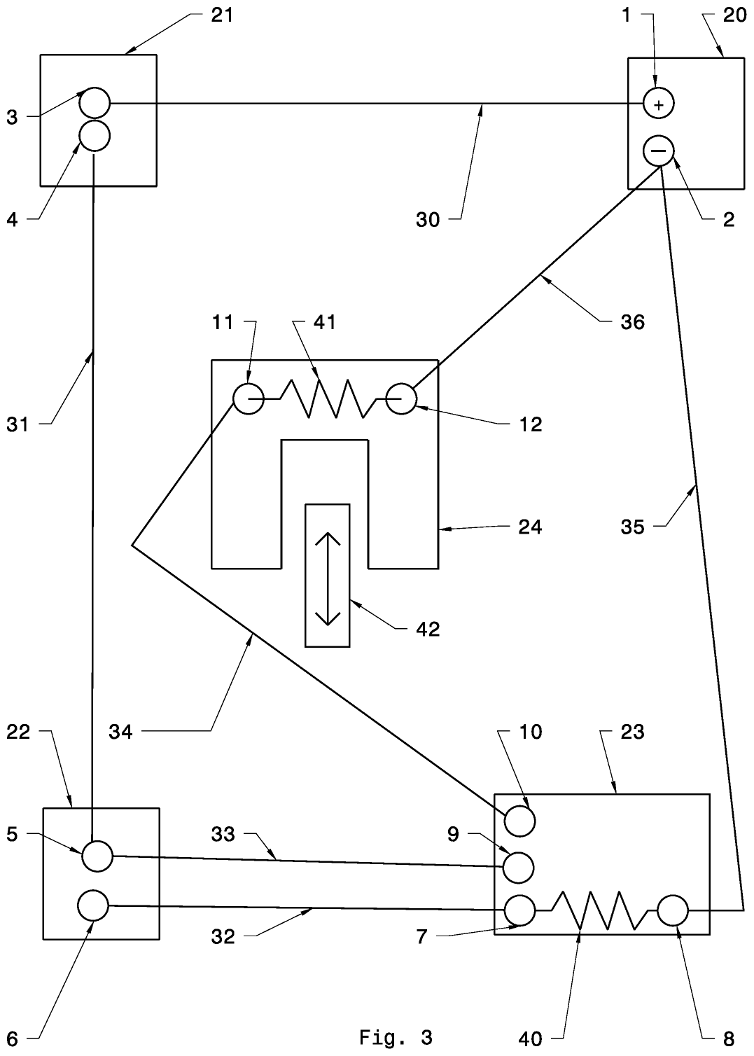

FIG. 3/3

Thermostat (21) calling cooling. Current flows from power source (20) terminal (1) to thermostat (21) terminal (3) Thermostat (21) terminals (3) and (4) make a contact Current flows from terminal (4) through wire (31) to delay make timer (22), terminal (5). Terminal (5) and terminal (6) are open do to pre-set time limit. Power flow stops at the terminal (5) on the timer. There is no current flow between Terminals (7) and (8), (10) and (9), (11) and (12), (12) and (2). On the solenoid (24) rod (42) drops in down position.

1. A automatic timer for dispenser, contain and comprising: a power source to output a current.

2. The automatic timer for dispenser of claim 1, further comprising: a delay on make timer.

3. The automatic timer for dispenser of claim 2, further comprising: The delay on make timer contacts are normally closed.

4. The automatic timer for dispenser of claim 3, further comprising: malty functional relay is now activated.

5. The automatic timer for dispenser of claim 4, further comprising: malty functional relay contacts are now closed.

6. The automatic timer for dispenser of claim 5 further comprising: solenoid is now activated.

7. The automatic timer for dispenser of claims 6, further comprising: rod in the solenoid is now in up position.

8. The automatic timer for dispenser of claim 7, further comprising: delay on make timer pre-set time, contacts are now open.

9. The automatic timer for dispenser of claim 8, further comprising: all contacts are now open.

10. The automatic timer for dispenser of claim 9, further comprising: the rod is now in down rest position.
